(19) United States
(12) Patent Application Publication
(10) Pub. No.: US 2015/0209404 A1
(43) Pub. Date: Jul. 30, 2015

(54) CELLULAR BLOOD MARKERS FOR EARLY DIAGNOSIS OF ALS AND FOR ALS PROGRESSION

(71) Applicant: YEDA RESEARCH AND DEVELOPMENT CO. LTD., Rehovot (IL)

(72) Inventors: Michal EISENBACH-SCHWARTZ, Rehovot (IL); Ester YOLES, Rehovot (IL); Hadas SCHORI, Rehovot (IL)

(21) Appl. No.: 14/677,123

(22) Filed: Apr. 2, 2015

Related U.S. Application Data
(62) Division of application No. 13/583,790, filed on May 23, 2013, now abandoned.
(60) Provisional application No. 61/312,335, filed on Mar. 10, 2010.

(51) Int. Cl. A61K 38/07; G01N 33/569; A61K 31/4985; A61K 31/53; A61K 31/7068; A61K 31/519

(52) U.S. Cl. CPC A61K 38/07; A61K 31/7068; A61K 31/519; A61K 31/4985; A61K 31/53; G01N 33/56972; G01N 2800/52; G01N 2333/70553; G01N 2333/70596

(57) ABSTRACT

The present invention provides methods for early diagnosis of amyotrophic lateral sclerosis (ALS) and for determining the efficacy of a treatment for ALS in an ALS patient, i.e., monitoring ALS progression, utilizing cellular blood markers; as well as kits for carrying out these methods.

### TECHNICAL FIELD

The present invention relates to methods for early diagnosis of amyotrophic lateral sclerosis (ALS) and for monitoring ALS progression, as well as to methods for treatment of said disease.

### BACKGROUND ART

The immune system is the body's natural mechanism for tissue healing and regeneration in all tissues. However, the presence and activity of peripheral immune cells in the central nervous system (CNS) was long considered to be undesirable because of the immune privileged nature of the CNS and the low tolerability of the brain to defensive battle (Gendelman, 2002). Yet, even though inflammation is considered to exacerbate CNS damage, anti-inflammatory agents have failed to show any significant benefit in numerous clinical trials (Anti-inflammatory drugs fall short in Alzheimer's disease, Nat Med., 2008; Etminan et al., 2008). An emerging understanding of the role of the immune system in regulating neurotoxicity (Marchetti et al., 2005; Cardona et al., 2006) has suggested that the situation is not so simple, with a balance between beneficial and detrimental effects of the immune system. More focused approaches to immune system modulation might be more successful than broad anti-inflammatory therapies.

“Protective autoimmunity” is a concept formulated by Prof. Michal Schwartz during the last decade. In response to injury, effector T-cells (T-eff) directed to self-antigens (autoimmune T-cells) are activated as part of a reparative response (Rapalino et al., 1998; Hauben et al., 2000; Hauben et al., 2003; Schwartz and Hauben, 2002; Moalem et al., 1999; Yoles et al., 2001; Kipnis et al., 2001; Schwartz et al., 2003), but this activity is tightly regulated by regulatory T cells (T-reg) (Taams and Akbar, 2005) as part of a mechanism to control autoimmune disease (Kipnis et al., 2002; Schwartz and Kipnis, 2002). Following CNS damage, exposed antigens from the damaged tissue activate T-eff in the peripheral lymphoid tissues. As the first stage of repair, these cells migrate and home specifically to the damaged tissue where they interact with local antigen presenting cells, resulting in secretion of growth factors, removal of dying neurons and detoxification of the environment (Shaked et al., 2004; Shaked et al., 2005). The timing, intensity and duration of this orchestrated immune response critically affect the ability of the milieu to support cell survival and regeneration (Nevo et al., 2003; Schwartz, 2002).

Amyotrophic lateral sclerosis (ALS), also known as Lou Gehrig's disease, is the most devastating adult-onset neurodegenerative disease, characterized by rapidly progressive failure of the neuromuscular system, resulting from degeneration and cell death of motor neurons in the spinal cord, brain stem and motor cortex, and leading to paralysis and death, usually within 3-5 years. While the majority of ALS cases are sporadic, about 5-10% of them are inherited, with the most abundant mutation occurring in the superoxide dismutase (SOD1) gene (Rosen, 1993). In both the sporadic and familial forms, disease progression is attributed to selective death of motor neurons in the spinal cord, with evidence for local neuroinflammation and acquisition of a cytotoxic phenotype by the microglia (Boillee et al., 2006; Clement et al., 2003; Gowing et al., 2008; Beers et al., 2006); however, it is still unclear what factor triggers the onset of the disease and what processes underlie the speedy propagation of motor neuron damage. Yet, current evidence suggests that regardless of the primary initiating event, progression of motor neuron damage involves activation of microglia, which produce neurotoxic factors as part of a vicious cycle (Sargsyan et al., 2005; Moisse and Strong, 2006). Post-mortem examination of spinal cords of ALS patients revealed a strong proinflammatory, neurotoxic immune cell profile (Graves et al., 2004) in the vicinity of degenerating motor neurons. Signs of an inflammatory response in the CNS at all stages of the disease were also described in mouse and rat models of ALS (carrying a transgene encoding mutant human SOD1); even before the onset of clinical signs of motor neuron injury, microglia are in an early state of activation, and levels of inflammatory mediators such as IL-1 are elevated. With the onset of symptoms and motor neuron death, microglia become chronically activated and produce TNF-α, a proinflammatory mediator.

In ALS, damage often starts focally, reflecting damage to a localized group of motor neurons, and spreads ‘like a brush fire’ to involve contiguous groups of motor neurons. It has been recently suggested that damage spreads through activation of microglia with the attendant release of neurotoxic factors. The spread of damage occurs when the “protective immunity” fails as a result of insufficient T-cell immunity, uncontrolled immunity (inflammation) or, paradoxically, immune deficiency.

Currently there is no effective treatment to ALS and moreover, there is difficulty in correctly diagnosing the patient at an early phase of the disease.

### SUMMARY OF INVENTION

In one aspect, the present invention relates to a method for diagnosing the likelihood of ALS in a tested individual, comprising:
(i) measuring the level of at least one cell type selected from regulatory T-cells, gamma-delta T-cells, pro-inflammatory monocytes, myeloid derived suppressor cells or natural killer cells in a peripheral blood sample obtained from said individual;
(ii) comparing the level measured for each one of said at least one cell type with a reference level representing a range level of each one of said cell types, respectively, in blood samples of age-matched controls, thus obtaining a test profile expressing a level of each one of said at least one cell type in the blood sample of said individual relative to the level of each one of said at least one cell type, respectively, in blood samples of age-matched controls; and
(iii) comparing said test profile with a reference profile expressing a representative relative level of each one of said at least one cell type in ALS patients,

wherein a significant similarity between said test profile and said reference profile indicates that said individual has a higher likelihood of having ALS than said age-matched controls.

In another aspect, the present invention relates to a method for determining the efficacy of a treatment for ALS in an ALS patient, said method comprising:
(i) measuring the level of at least one cell type selected from regulatory T-cells, gamma-delta T-cells, myeloid derived suppressor cells or natural killer cells in a peripheral blood sample obtained from said patient at two consecutive instants, the earlier of said instants is prior to or during said treatment and the later of said instants is during said treatment; and
(ii) comparing the levels measured for each one of said at least one cell type at said two instants,

wherein an alteration of the level measured for one or more of said at least one cell type at said later instant compared with the level measured for said cell type at said earlier instant towards a predetermined level representing a range level of said cell type in blood samples of healthy controls is correlated with the efficacy of said treatment.

In a further aspect, the present invention relates to a method for treatment of an ALS patient comprising administering to said patient an effective amount of an agent capable of reducing myeloid derived suppressor cell level in peripheral blood.

In still another aspect, the present invention relates to a method for treatment of an ALS patient comprising administering to said patient an effective amount of an agent capable of inducing migration of immature myeloid cells from the peripheral blood to the injured spinal cord of said patient upon stimulation with chemokine interleukin 8 (CXCL8) or chemokine (C—C motif) ligand 2 (CCL2).

In yet another aspect, the present invention relates to a method for treatment of an ALS patient comprising injecting into the cerebral spinal fluid (CSF) of said patient an effective amount of autologous myeloid derived cells.

In still a further aspect, the present invention provides a kit for diagnosing the likelihood of ALS in a tested individual; or for determining the efficacy of a treatment for ALS in an ALS patient, said kit comprising:
(i) a list of cell types selected from regulatory T-cells, gamma-delta (γδ) T-cells, pro-inflammatory monocytes, myeloid derived suppressor cells (MDSCs), or natural killer cells;
(ii) antibodies against each one of said cell types;
(iii) reagents for detecting said antibodies;
(iv) a list of reference levels representing range levels of said cell types in blood samples of age-matched controls;
(v) optionally a reference profile expressing a representative relative level of each one of said cell types in blood samples of ALS patients; and
(vi) instructions for use.

### BRIEF DESCRIPTION OF DRAWINGS

FIG. 1 shows that the level of CD11b⁺/CD14⁻ myeloid derived suppressor cells (MDSCs) in peripheral blood is significantly elevated in ALS patients. Fresh whole blood samples of ALS patients, Alzheimer's (AD) patients, age-matched controls and young controls (n=7, 12, 10 and 6, respectively) were stained with monoclonal antibodies against CD14 and CD11b, and the dots represent the percentage of CD11b⁺/CD14⁻ cells out of the total monocyte population for each patient, determined by FACS. As shown, the percentage of CD11b⁺/CD14⁻ cells out of total monocytes in ALS patients was significantly higher compared to age-matched controls (P<0.004; Student's t test), young controls (P<0.003; Student's t test) and Alzheimer's disease patients (P<0.001; Student's t-test).

FIG. 2 shows that the level of Lin⁻/HLA-DR⁻/CD33⁺ MDSCs in peripheral blood is significantly elevated in ALS patients. Fresh whole blood samples of ALS patients and age-matched controls (n=15 and 10, respectively) were stained with monoclonal antibodies against Lin, HLA-DR and CD33, and the dots represent the percentage of Lin⁻/HLA-DR⁻/CD33⁺ cells out of the total monocyte population for each patient, determined by FACS. As shown, the percentage of Lin⁻/HLA-DR⁻/CD33⁺ myeloid cells out of total monocytes in ALS patients was significantly higher compared to age-matched controls (P<0.02; Student's t-test).

FIG. 3 shows that the percentage of γδ T cells out of total CD3 cells in peripheral blood mononuclear cells (PBMCs) is significantly elevated in ALS patients. Fresh whole blood samples of ALS patients and healthy control (n=7 in each group) were double-stained with monoclonal antibodies against CD3 and with monoclonal antibodies γδ T cell receptor, and the dots represent the percentage of γδ T cells out of total CD3 cells, determined by FACS. As shown, the percentage of γδ T cells out of total CD3 cells in ALS patients was significantly higher compared to healthy controls (P<0.004; Student's t test).

### DETAILED DESCRIPTION OF THE INVENTION

The present invention is based on a concept according to which CNS pathologies emerge following a long stage of struggle between the disease pathology and the attempts of the immune system to fight it off. In particular, this concept describes a multi-step process that is, in fact, very similar to the process by which the body prevents cancer, i.e., the process termed “tumor immunoediting”, characterized by the three consecutive phases “elimination”, “equilibrium” and “escape” (“the three Es”, for extensive reviews see Dunn et al., 2002, and Smyth et al., 2006).

In general, little is known about the dialogue between the immune system and the diseased CNS at the pre-onset stage, i.e., prior to the emergence of the clinical symptoms. Thus, in order to gain insight into the possible stages at which failure of the immune system could take place, we examined whether the principles that guide immune surveillance in the context of tumors are also applicable to neurodegenerative diseases, focusing particularly on amyotrophic lateral sclerosis (ALS).

Elimination:

Until the last decade, it was generally believed that any acute or chronic disorder of the CNS must be repaired by the CNS tissue alone, and that any immune-cell activity at the site of damage would be insignificant at best or harmful at worst. We suggest that, as in the elimination phase of tumor immunoediting, any deviation from homeostasis in the CNS triggers a cascade of immune responses, which orchestrates a process that restores homeostasis and thereby limits the damage and facilitates repair. According to this view, immediately after the occurrence of the deviation, a variety of toxic mediators emerge. As a result, the local innate immune cells (microglia) are activated by the dying cells and/or by the self-compounds that exceed physiological levels and become toxic (Schwartz et al., 2003; Shaked et al., 2004). Thus, the surrounding still-healthy neurons are subjected to a threatening milieu that, if not corrected immediately, will affect these cells as well (a phenomenon that is known as spread of damage). The microglia release chemokines and act to clear the damaged site from the debris and toxic self-compounds. Subsequently, antigens released from the damaged tissue are carried to the draining lymph nodes by local antigen presenting cells (APCs), which in turn activate T cells that specifically recognize self-antigens released at the damaged site (Karman et al., 2004; Ling et al., 2006). Importantly, such self-antigens, by themselves, are not necessarily pathogenic, as is the case of neoantigens in tumors. The CNS-specific T cells home to the damaged site, where they engage in cross talk with local APCs such as microglia and infiltrating macrophages (Schori et al., 2001). As a result of this T cells/APCs interaction, cytokines and chemokines are released from both the T cells and the APCs, inducing an infiltration of a second wave of bone marrow derived monocytes. These monocytes, which are now exposed to the T cell regulated immunological milieu at the site of injury, produce growth factors such as insulin-like growth factor I (IGF-I) and brain-derived neurotrophic factor (BDNF), which contribute to neuronal survival, i.e., prevent spread of damage, and to tissue repair by endogenous stem/progenitor cells (Ziv et al., 2006; Ziv et al., 2007). This series of events, which occurs following CNS insult or deviation from homeosatasis, may represent an elimination phase analogous to the one observed in tumor immunology. By nature, acute insults in the CNS result in a steady state; a scar tissue composed of glial cells and extracellular-matrix proteoglycans, e.g., chondroitin sulfate proteoglycan (CSPG), confine the site of injury, while spared cells and newly formed neurons and glial cells reside at the margin of the quarantined injury site (Rolls et al., 2004). Thus, as far as immune system activity is concerned, acute insults are resolved at the elimination phase.

Equilibrium:

We suggest that in cases of chronic neuropathological conditions, the failure to completely eliminate the threat and restore homeostasis leads to conditions that appear similar to those found in the equilibrium phase of the immune response against tumors, during which the disease is dormant, i.e., symptom-free. Such situations may occur in chronic neurodegenerative disorders such as ALS. Although animal studies have shown that in all these pathologies, once the clinical symptoms emerge, immune activity affects the course of the disease (Butovsky et al., 2006; Beers et al., 2006; Laurie et al., 2007), we suggest that the immune system struggles with early manifestations of these diseases long before they become symptomatic. In this way, immune activity could maintain neuropathological disorders in a dormant state for years, very much like it does in cancer. The point at which clinical symptoms appear represents the beginning of what could be considered as the parallel to the ‘escape’ phase, which could be an outcome of either suppression of the immune response imposed by the dying neurons, or a local innate inflammatory response.

Escape:

In contrast to tumor immunoediting, in neurodegenerative disorders the immune system does not impose true selection forces on the factor/s that induce the damage. This distinction is integral to the fact that in cancer, immune activity is required to selectively kill cells, while in neurodegeneration, immune activity is needed to remove the emerging threats and to promote cell survival and renewal in a non-selective manner. Nevertheless, during the course of a neurodegenerative disease, toxicity mediators, damaging factors and dying cells can escape immune surveillance. As in tumor escape, both suppression of adaptive immunity and overwhelming local inflammation can lead to escalation of a neurodegenerative process.

A neurodegenerative disease in which escape from immune surveillance could take place is ALS, which predominantly affects motor neurons. Most of the knowledge about pathophysiological mechanisms of ALS derives from experiments carried out in a strain of transgenic mice that spontaneously develop an ALS-like disease. These mice express the mutant human Cu²⁺/Zn²⁺ superoxide dismutase (SOD1) protein, which corresponds to 10-15% of the familial ALS cases, representing 5-10% of all ALS cases. Although extensive studies have been performed on ALS mice, it is still not clear how the mutant SOD1, which is ubiquitously expressed in all tissues, causes specific motor neuron degeneration.

In support for a role of immune cells in ALS disease progression, are several studies showing that replacing the bone marrow of ALS mice with bone marrow derived from healthy animals increases life-expectancy (Simard et al., 2006; Huang et al., 2006; Corti et al., 2004). An elegant demonstration of the effect of CNS-resident microglia in ALS disease progression comes from an experiment in which bone marrow from wild type mice was transplanted into neonatal ALS mice, which also suffer from a complete immune deficiency (Beers et al., 2006). In these mice, the neonatal bone marrow transplantation resulted in population of the brain with microglia that did not express the mutant SOD1 form. This manipulation slowed motor neuron loss and prolonged disease duration and survival, when compared with mice receiving bone marrow transplantation from ALS mice, i.e., mice containing the mutant SOD1. Importantly, transplantation of bone marrow from ALS mice into wild mice did not induce any signs of neurodegeneration, indicating that microglia are affected by the SOD1 mutation in a way that causes exacerbation of the disease, but are not the primary damaging components.

The majority of studies suggest that microglia contribute to ALS progression by producing toxic inflammatory compounds. In vitro studies have shown that microglia from ALS mice produce higher levels of TNF-α when stimulated with lipopolysaccharide (LPS) compared to wild type microglia. A recent study found that mutant, but not wild type SOD1, is released from motor neurons, and can, by itself, activate microglia so as to become detrimental (Weydt et al., 2004). Collectively, the findings from ALS mice suggest that escape from immune surveillance can be achieved, at least in part, through alteration of the microglial phenotype. Microglial activation has been demonstrated in the brain and spinal cord of ALS patients and in the spinal cord of ALS mice. Moreover, relative to wild type mice, elevated levels of monocyte chemoattractant protein-1 (MCP-1) were found in ALS mice as early as 15 days of age; and by 39 days of age, CD68⁺ cells (presumably dendritic cells) were found in the spinal cord of ALS mice (Henkel et al., 2004). These findings suggest that the damage begins to develop very early in life, much before clinical signs are manifested. Yet, although some signs of immune activity are evident before the paralyzing symptoms appear, significant infiltration of bone marrow-derived monocytes and T cells occurs only at very late stages of the disease (Kunis, Bukshpein and Schwartz, unpublished results), suggesting that the death of the motor neurons is not sufficient to trigger the adaptive immune response that is required for the recruitment of peripheral myeloid-derived cells needed for defense, or that this response is actively suppressed.

Preliminary studies conducted in accordance with the present invention and described hereinafter have shown specific and consistent changes in the levels of certain myeloid derived suppressor cells (MDSCs), more particularly CD11b⁺/CD14⁻ and Lin⁻/HLA-DR⁻/CD33⁺ cells, as well as of gamma-delta (γδ) T-cells, in peripheral blood samples of ALS patients, compared with those measured in peripheral blood samples of age-matched controls. The alteration in the level of said MDSCs has not been observed in individuals suffering from other neurodegenerative diseases such as Alzheimer's disease. Furthermore, in contrast to other neurodegenerative diseases such as Alzheimer's disease, no alteration has been observed in the level of the pro-inflammatory monocytes CD14⁺/CD16⁺ cells in blood samples of ALS patients, as shown in Table 1 below. These findings indicate that specific changes in the level of certain T-cell or monocyte subsets such as those mentioned above can be used, either separately or in combination with each other or with other markers, as blood markers for diagnosis of ALS and for monitoring ALS progression and treatment efficacy.

TABLE 1
CD14⁺/CD16⁺ cell level in ALS and Alzheimer's
disease patients vs. controls

| | n | Average | SD | Min | Median | Max |
|---|---|---|---|---|---|---|
| Healthy controls | 14 | 10.5 | 5.78 | 2.40 | 9.7 | 20.4 |
| Alzheimer's patients | 15 | 16.3 | 8.70 | 3.20 | 17.4 | 34.9 |
| ALS patients | 7 | 7.8 | 3.90 | 2.50 | 8.1 | 14.7 |

In one aspect, the present invention thus relates to a method for diagnosing the likelihood of ALS in a tested individual, comprising:
(i) measuring the level of at least one cell type selected from regulatory T-cells, gamma-delta T-cells, pro-inflammatory monocytes, myeloid derived suppressor cells or natural killer cells in a peripheral blood sample obtained from said individual;
(ii) comparing the level measured for each one of said at least one cell type with a reference level representing a range level of each one of said cell types, respectively, in blood samples of age-matched controls, thus obtaining a test profile expressing a level of each one of said at least one cell type in the blood sample of said individual relative to the level of each one of said at least one cell type, respectively, in blood samples of age-matched controls; and
(iii) comparing said test profile with a reference profile expressing a representative relative level of each one of said at least one cell type in ALS patients,

wherein a significant similarity between said test profile and said reference profile indicates that said individual has a higher likelihood of having ALS than said age-matched controls.

The term “regulatory T-cells”, as used herein, refers to a specialized subpopulation of T cells, also known as suppressor T cells, which act to suppress activation of the immune system and thereby maintain immune system homeostasis and tolerance to self-antigens. Regulatory T cells come in many forms, including those that express the CD8 transmembrane glycoprotein (CD8⁺ T cells), those that express CD4, CD25 and FoxP3 (CD4⁺CD25⁺ regulatory T cells) and other T cell types having suppressive function. A non-limiting example of regulatory T cells according to the present invention is CD4⁺/CD25⁺/FoxP3 cells.

The term “gamma-delta (γδ) T-cells”, as used herein, refers to a small subset of T cells possessing a distinct T cell receptor (TCR) on their surface. In contrast to a majority of T cells in which the TCR is composed of two glycoprotein chains designated α- and β-TCR chains, the TCR in γδ T cells is made up of a γ-chain and a δ-chain. These cells were shown to play a role in immunosurveillance and immunoregulation (Girardi, 2006), and were found to be an important source of IL-17 (Roark et al., 2008) and to induce robust CD8⁺ cytotoxic T cell response (Brandes et al., 2009).

The term “pro-inflammatory monocytes”, as used herein, refers to a non-classical type of monocytes characterized by low-level expression of CD14 and additional co-expression of the CD16 receptor (CD14⁺/CD16⁺ monocytes), which develop from the CD14⁺⁺ monocytes.

The term “myeloid derived suppressor cells (MDSCs)”, as used herein, refers to a heterogeneous population of cells consisting of myeloid progenitor cells and immature myeloid cells (IMCs). In healthy individuals, IMCs that are quickly generated in the bone marrow differentiate into mature granulocytes, macrophages or dendritic cells (DCs). Interference with the differentiation of IMCs into mature myeloid cells results in the expansion of MDSC population. Accumulating evidence has shown that MDSCs contribute to the negative regulation of immune responses during cancer and other diseases. In human cancer, a subset of myeloid cells was found to have significantly increased arginase activity, which down-regulates expression of the T cell receptor CD3-ζ chain; and to suppress T cell proliferation, suggesting that these cells may mediate tumor-related immune suppression (Ochoa et al., 2007; Zea et al., 2005). Moreover, since it was shown that IL-13 plays a crucial role in MDSC suppressive activity (Beers et al., 2008), our suggestion that MDSC activity is involved in disease progression is consistent with a report showing that the percentages of both CD4⁺IL-13⁺ and CD8⁺IL-13⁺ T cells in the blood of ALS patients are significantly higher than in healthy controls. The proportion of CD4⁺IL-13⁺ T cells was shown to have a significant negative correlation with the ALS functional rating scale scores, and a significant positive correlation with the rate of disease progression (Chiu et al., 2008).

Non-limiting examples of MDSCs according to the present invention include CD11b⁺/CD14⁻, CD11b⁺/CD14⁻/CD15⁺, CD11b⁺/CD14⁺/CD15⁺, Lin⁻/DR⁻, Lin⁻/DR⁻/CD33⁺, CD34⁺/CD33⁺/CD13⁺, ARG⁺/CD14⁺, CD34⁺/Lin⁻/DR⁻/CD11b⁺/CD15⁺, CD14⁺/HLA-DR⁻/low, and Lin⁻/HLA-DR⁻/low/CD11b⁺/CD33⁺ cells.

The term “natural killer (NK) cells”, as used herein, refers to a type of cytotoxic lymphocytes that constitute a significant component of the innate immune system, and play a major role in the rejection of tumors and cells infected by viruses by releasing small cytoplasmic granules of proteins that induce apoptosis in the target cells. These cells do not express TCR, Pan T marker CD3 or surface immunoglobulin B cell receptor, but they usually express the surface markers CD16 (FcγRIII) and CD56 in humans. Up to 80% of NK cells further express CD8. Non-limiting examples of natural killer cells according to the present invention include CD16⁺ and CD16⁺/CD56⁺ cells.

The level of each one of the cell types or subsets defined above, in the peripheral blood sample tested, can be measured utilizing any suitable technique known in the art, e.g., as described in Materials and Methods hereinafter.

The level measured for each one of the cell types or subsets tested, according to step (i) of the diagnosing method of the present invention, is compared with a reference level representing a range level of said cell type or subset in blood samples of age-matched controls, i.e., a group of healthy individuals in the same age-group as the tested individual. This range level is derived from the available medical knowledge and represents the normal range level for the specific cell type or subset tested in blood samples of age-matched controls.

According to step (ii) of this method, after comparing the level measured for each one of the cell types or subsets tested with the reference level, i.e., the normal range level, thereof, a test profile is obtained, expressing the level of each one of the cell types of subsets tested in the blood sample obtained from the tested individual relative to the level of each one of these cell types or subsets, respectively, in blood samples of age-matched controls.

The term “test profile”, as used herein, refers to a profile showing the level of each one of the cell types or subsets measured according to the method of the present invention in the blood sample obtained from the tested individual relative to the reference level thereof in blood samples of age-matched controls. According to step (i) of this method, the level of at least one cell type or subset is measured, and therefore, the test profile obtained expresses the level of at least one, but preferably two, three, four, five, six, or more cell types or subsets, as defined above.

The relative level of each one of the cell types or subsets measured is represented in the test profile by “increase”, indicating that the level of said cell type or subset in the blood sample obtained from the tested individual is increased compared with the upper limit of the normal range level thereof, i.e., the range level of said cell type or subset in blood samples of age-matched controls, by at least about 10%, preferably at least about 20%, more preferably at least about 30%, 40%, or 50%; “decrease”, indicating that the level of said cell type or subset in the blood sample obtained from the tested individual is decreased compared with the lower limit of the normal range level thereof by at least about 10%, preferably at least about 20%, more preferably at least about 30%, 40%, or 50%; or “no change”, indicating that the level of said cell type or subset in the blood sample obtained from the tested individual is neither increased nor decreased as defined above, i.e., within or close to the normal range level thereof.

According to step (iii) of the diagnosing method of the present invention, in order to determine whether the tested individual has a higher likelihood of having ALS, the test profile obtained in step (ii) is compared with a reference profile expressing a representative relative level of each one of the cell types or subsets measured in ALS patients. The term “reference profile”, as used herein, refers to a predetermined profile established for a group of ALS patients, based on the level measured for each one of the cell types or subsets in blood samples obtained once in a while from each one of these patients, showing the representative relative level, in terms of “increased”, “decreased” and “no change” as defined above, of each one of the cell types or subsets measured in the blood samples obtained from these ALS patients.

Although the reference profile according to the method of the present invention is predetermined, it should be understood that this profile might be established using any suitable algorithm. For example, the representative relative level of a certain cell type or subset measured is represented by “increase”, indicating that the level of said cell type or subset in a majority of the ALS patients in the group is increased compared with the normal range level of said cell type or subset; “decrease”, indicating that the level of said cell type or subset in a majority of the ALS patients is decreased compared with the normal range level of said cell type or subset; or “no change”, indicating that the level of said cell type or subset in a majority of the ALS patients is neither increased nor decreased, as defined above, compared with the normal range level of said cell type or subset.

The phrase “significant similarity between said test profile and said reference profile” refers to a situation in which the pattern of alterations observed in the test profile with respect to the majority of the cell types or subsets included in the profile is identical to the pattern of alterations indicated with respect to these cell types or subsets in the predetermined reference profile established for a group of ALS patients. In fact, the likelihood that the tested individual has ALS is considered to increase with the increase in the number of cell types of subsets, which are altered in the test profile in the direction defined by the reference profile, wherein a total similarity between the profiles indicates a very high likelihood that the tested individual has ALS. It should be understood that in cases levels of one or two cell types or subsets only are measured, a decision whether the tested individual has a likelihood of having ALS can be made only if a total similarity between the two profiles is observed.

In certain embodiments, the cell types the levels of which are measured in step (i) of the diagnosing method of the invention are selected from γδ T-cells, pro-inflammatory monocytes, or MDSCs, as defined above.

In particular embodiments, the predetermined reference profile expressing a representative relative level of each one of the cell types measured in ALS patients comprises an increase in the level of γδ T-cells; an increase in the level of at least one type of MDSCs selected from CD11b⁺/CD14⁻, CD11b⁺/CD14⁻/CD15⁺, CD11b⁺/CD14⁺/CD15⁺, Lin⁻/DR⁻, Lin⁻/DR⁻/CD33⁺, CD34⁺/CD33⁺/CD13⁺, ARG⁺/CD14⁺, CD34⁺/Lin⁻/DR⁻/CD11b⁺/CD15⁺, CD14⁺/HLA-DR⁻/low, or Lin⁻/HLA-DR⁻/low/CD11b⁺/CD33⁺; and no change in the level of CD14⁺/CD16⁺ cells.

In more particular embodiments, the predetermined reference profile comprises an increase in the level of γδ T-cells; an increase in the level of CD11b⁺/CD14⁻ and/or Lin⁻/DR⁻/CD33⁺ MDSCs; optionally an increase in the level of at least one, two, or three further types of MDSCs selected from CD11b⁺/CD14⁻/CD15⁺, CD11b⁺/CD14⁺/CD15⁺, Lin⁻/DR⁻, CD34⁺/CD33⁺/CD13⁺, ARG⁺/CD14⁺, CD34⁺/Lin⁻/DR⁻/CD11b⁺/CD15⁺, CD14⁺/HLA-DR⁻/low, or Lin⁻/HLA-DR⁻/low/CD11b⁺/CD33⁺; and no change in the level of CD14⁺/CD16⁺ cells.

In a certain particular embodiment, the predetermined reference profile comprises an increase in the level of γδ T-cells; an increase in the levels of both CD11b⁺/CD14⁻ and Lin⁻/DR⁻/CD33⁺ MDSCs; and no change in the level of CD14⁺/CD16⁺ cells.

In the studies described in the Examples hereinafter, certain immunological alterations have been observed in the blood of ALS patients compared with that of age- and gender-matched volunteers that do not suffer from ALS. In particular, venous blood was obtained from ALS patients and from controls, and blood samples were characterized by whole blood flow cytometry for the level of certain mononuclear cell subsets or the expression of specific membrane markers. In general, the average percentage of CD14⁺ monocytes was 16.6±6.3 and 18.9±4.3 in controls and ALS blood samples, respectively (Student t-test p=0.35), i.e., no difference was found in the percentage of monocytes within the mononuclear cell population between the groups. However, Example 1 shows a dramatic elevation in the percentage of cells expressing the membrane markers CD11b⁺/CD14⁻, an immature monocyte phenotype associated with MDSCs, in the blood of ALS patients compared with that of their age-matched controls; Example 2 shows that the percentage of cells expressing the membrane markers Lin⁻/DR⁻/CD33⁺ out of total peripheral blood mononuclear cells (PBMCs) in the blood of ALS patients is significantly higher than that in their age-matched controls; and Example 3 shows that the percentage of gamma-delta T cells out of total CD3 cells in the blood of ALS patients is significantly higher than that in their age-matched controls.

In a certain particular embodiment, the cell types the levels of which are measured in step (i) are thus γδ T-cells, CD11b⁺/CD14⁻ cells, Lin⁻/DR⁻/CD33⁺ cells, and CD14⁺/CD16⁺ cells; and the reference profile expressing a representative relative level of each one of said cell types in ALS patients comprises an increase in the level of gamma-delta T-cells, an increase in the level of CD11b⁺/CD14⁻ cells, an increase in the level of Lin⁻/DR⁻/CD33⁺ cells, and no change in the level of CD14⁺/CD16⁺ cells.

In view of all the aforesaid, the present invention particularly provides a method for diagnosing the likelihood of ALS in a tested individual, comprising:
(i) measuring the level of the cell types gamma-delta T-cells, CD11b⁺/CD14⁻ cells, Lin⁻/DR⁻/CD33⁺ cells and CD14⁺/CD16⁺ cells in a peripheral blood sample obtained from said individual; and
(ii) comparing the level measured for each one of said cell types with a reference level representing a range level of each one of said cell types, respectively, in blood samples of age-matched controls,

wherein an increase in the level of gamma-delta T-cells, an increase in the level of CD11b⁺/CD14⁻ cells, an increase in the level of Lin⁻/DR⁻/CD33⁺ cells and no change in the level of CD14⁺/CD16⁺ cells indicate that said individual has a higher likelihood of having ALS than said age-matched controls.

It is expected that alterations observed in the level of certain cell types or subsets measured in a blood sample of a patient suffering from progressive ALS at a first instant will be weaker, i.e., less pronounced than those measured in a blood sample taken from the same patient, at a second instant that is about 1, 2, 3, 4, 5, 6 months or more later than the first one. In other words, it can be assumed that a progression of the disease would be reflected in the levels measured for one or more of the cell types or subsets tested, wherein the differences between the levels measured at the later instant for at least one of the cell types or subsets tested and the normal range levels of said cell type or subset will be significantly greater than those obtained for said cell types or subsets at the earlier instant. Similarly, it may be expected that a moderation in at least some of the alterations observed in the first instant will be noticed at the later instant in case an effective therapeutic treatment for ALS is given to said patient.

In another aspect, the present invention thus relates to a method for determining the efficacy of a treatment for ALS in an ALS patient, comprising:
(i) measuring the level of at least one cell type selected from regulatory T-cells, gamma-delta T-cells, myeloid derived suppressor cells or natural killer cells in a peripheral blood sample obtained from said patient at two consecutive instants, the earlier of said instants is prior to or during said treatment and the later of said instants is during said treatment; and
(ii) comparing the levels measured for each one of said at least one cell type at said two instants,

wherein an alteration of the level measured for one or more of said at least one cell type at said later instant compared with the level measured for said cell type at said earlier instant towards a reference level representing a range level of said cell type in blood samples of age-matched controls is correlated with the efficacy of said treatment.

In contrast to the diagnosing method described above, in which the level of certain cell types or subsets in a blood sample obtained from a tested individual is compared with the level of those cell types or subsets in blood samples of age-matched controls, in this method, in which the efficacy of a treatment for ALS in an ALS patient is determined, the level of such cell types or subsets in a peripheral blood sample obtained from an ALS patient is measured at two consecutive instants and are then compared so as to evaluate the progression of the disease or, alternatively, the efficacy of an ALS treatment given to said patient.

The phrase “a range level of said cell type in blood samples of age-matched controls”, as used herein, refers to the normal range level for a specific cell type or subset in blood samples of age-matched controls, as defined above.

The phrase “an alteration of the level measured for one or more of said at least one cell type at said later instant compared with the level measured for said cell type at said earlier instant towards a reference level representing a range level of said cell type in blood sample of age-matched controls”, as used herein, refers to any case in which the difference between the level measured at the earlier instant for at least one of the cell types or subsets tested and the normal range level of said cell type or subset is significantly greater that that obtained for said cell type or subset at the later instant when compared with the normal range level of said cell type or subset. An alteration of the level measured for a certain cell type or subset at said later instant compared with the level measured for said cell type or subset at said earlier instant towards the normal range level of said cell type or subset may thus be defined as a significantly less pronounced increase in cases wherein the relative level of said cell type or subset at the earlier instant is represented by “increase”, or a significantly less pronounced decrease in cases wherein the relative level of said cell type or subset at the earlier instant is represented by “decrease”, as defined above respectively.

According to this method, the earlier of said instants is prior to or during said treatment and the later of said instants is during said treatment. Thus, in certain embodiments, the earlier of said two consecutive instants is prior to said treatment and the later of said instants is following about 1, 2, 3, 4, 5, 6 months or more of said treatment. In other embodiments, the earlier of said two consecutive instants is at any point in time during said treatment and the later of said instants is about 1, 2, 3, 4, 5, 6 months or more after the earlier of said two instants.

As described above, in contrast to certain neurodegenerative diseases such as Alzheimer's disease, no alteration has been observed in the level of the pro-inflammatory monocytes CD14⁺/CD16⁺ cells in peripheral blood samples of ALS patients compared with the normal range level of these cells. Therefore, while the level of these monocytes can be used, in combinations with the level of other cell types or subsets as defined above, for diagnosing the likelihood of ALS in a tested individual, the level of these specific monocytes has no importance in monitoring the progression of said disease or in determining the efficacy of a treatment for ALS in an ALS patient.

Nevertheless, when carrying out this method and as to guarantee that the levels measured for the various cell types or subsets tested at each one of the two consecutive instants are not influenced by an external factor such as inflammation and can thus be relied upon, it is recommended that at least one cell type or subset the level of which in ALS patients is within the normal range level thereof, is further tested and serves as a control.

The elevated level of cells reminiscent of myeloid suppressor cells in the blood of ALS patients might appear to contradict the chronic inflammation observed in the microenvironment of CNS lesions. Actually, the presence of high levels of suppressor cells in the periphery suppress recruitment of blood-derived monocytes, including those that locally become suppressor cells, into the site of local inflammation in the CNS. Recruitment of such monocytes depends upon activation of CNS specific T-cells (Shechter et al., 2009). MDSC infiltration into the CNS was also described as T-cell dependent in patients suffering from malignant glioma, leading to local inhibition of cytotoxic T-cell function. Indeed, any previous attempts to suppress systemic immune activity as means of curtailing the local response have failed, except in cases of systemic inflammation as a cause of such diseases, as is the case of autoimmune diseases including multiple sclerosis (MS). For example, both minocycline and daily Copaxone®, which are effective in treating MS, an inflammatory disease, failed and were even detrimental in ALS (Gordon et al., 2007).

The immunosuppression nature of the systemic immune response found here, coupled with a severe deficiency in newly-formed T cells found (Seksenyan et al., 2009), further support the contention that malfunction of the systemic immune response in ALS patients is a co-morbidity factor in the disease (Frey and Monu, 2008; Serafini et al., 2006a). It is postulated that the findings described above provide the missing link between the peripheral and local immune activity that may explain disease onset and progression. In view of that, we suggest that accumulation of toxic components such as oxygen radicals and neurotransmitters, i.e., glutamate, at the microenvironment of motor neurons in the spinal cord following excessive motor activity activates the microglia as the first step in restoration of homeostasis. It appears that in ALS, the local inflammation fails to recruit assistance from the adaptive immune system due to deficiency in newly formed T-cells that can be activated to recognize CNS antigens, and as a consequence, the neurotoxic inflammatory activity becomes chronic and spreads within the tissue. Chronic inflammation is one of the conditions known to increase the level of MDSCs, probably as part of homeostatic efforts to control inflammation. In ALS patients, the deficiency in adaptive immune activity also leads to reduction in MDSCs infiltration into the CNS. Thus, the local inflammation not only fails to evoke the proper peripheral neuroprotective immune response, but also actively suppresses it by systemic induction of MDSCs, eventually culminating in immune deficiency. Our results thus suggest a new approach of immune rejuvenation as a therapy in ALS, by viewing defects in immune function as a co-morbidity factor, and thus, as a potential target for therapeutic intervention.

In particular, in a further aspect, the present invention relates to a method for treatment of an ALS patient comprising administering to said patient an effective amount of an agent capable of reducing myeloid derived suppressor cell level in peripheral blood. Any agent capable of reducing myeloid derived suppressor cell level in a peripheral blood can be used, wherein examples of such agents, without being limited to, include gemcitabine, sildenafil, tadalafil and vardenafil (Suzuki et al., 2005; Serafini et al., 2006b).

In certain embodiments, this therapeutic method further comprises administering to the patient an effective amount of an agent capable of augmenting level of anti-self T-cells in a peripheral blood such as glatiramer acetate (Copaxone®, approved for treatment of relapsing-remitting MS), autologous T cells and/or activated T cells.

In still another aspect, the present invention relates to a method for treatment of an ALS patient comprising administering to said patient an effective amount of an agent capable of inducing migration of immature myeloid cells from the peripheral blood to the injured spinal cord of said patient upon stimulation with chemokine interleukin 8 (CXCL8) or chemokine (C—C motif) ligand 2 (CCL2).

In yet another aspect, the present invention relates to a method for treatment of an ALS patient comprising injecting into the cerebral spinal fluid (CSF) of said patient an effective amount of autologous myeloid derived cells. These cells are needed at the site of damage in the spinal cord and brain to modulate the distractive pro-inflammatory environment and to enhance the initiation of protective immune activity.

In still a further aspect, the present invention provides a kit for diagnosing the likelihood of ALS in a tested individual; or for determining the efficacy of a treatment for ALS in an ALS patient, said kit comprising:
(i) a list of cell types selected from regulatory T-cells, gamma-delta (γδ) T-cells, pro-inflammatory monocytes, myeloid derived suppressor cells (MDSCs), or natural killer cells;
(ii) antibodies against each one of said cell types;
(iii) reagents for detecting said antibodies;
(iv) a list of reference levels representing range levels of said cell types in blood samples of age-matched controls;
(v) optionally a reference profile expressing a representative relative level of each one of said cell types in blood samples of ALS patients; and
(vi) instructions for use.

The kit of the present invention can be used for carrying out both of the non-therapeutic methods described above, i.e., both the method in which the likelihood of ALS in a tested individual is diagnosed, and the method in which the efficacy of a treatment for ALS in an ALS patient is determined.

The kit of the invention comprises a list of cell types the levels of which are measured in a blood sample obtained from either an individual tested for ALS or an ALS patient receiving a treatment for ALS. The various categories of the cell types, i.e., regulatory T-cells, γδ T-cells, pro-inflammatory monocytes, MDSCs, and natural killer cells, are defined above.

In certain embodiments, the cell types listed are selected from γδ T-cells, pro-inflammatory monocytes, or MDSCs. In particular embodiments, the cell types listed are γδ T-cells; at least one type of MDSCs selected from CD11b⁺/CD14⁻, CD11b⁺/CD14⁻/CD15⁺, CD11b⁺/CD14⁺/CD15⁺, Lin⁻/DR⁻, Lin⁻/DR⁻/CD33⁺, CD34⁺/CD33⁺/CD13⁺, ARG⁺/CD14⁺, CD34⁺/Lin⁻/DR⁻/CD11b⁺/CD15⁺, CD14⁺/HLA-DR⁻/low, or Lin⁻/HLA-DR⁻/low/CD11b⁺/CD33⁺; and the pro-inflammatory CD14⁺/CD16⁺ cells. In more particular embodiments, the cell types listed are γδ T-cells; at least one type of MDSCs selected from CD11b⁺/CD14⁻, or Lin⁻/DR⁻/CD33⁺ MDSCs, preferably both CD11b⁺/CD14⁻, and Lin⁻/DR⁻/CD33⁺ MDSCs; optionally at least one, two, or three further types of MDSCs selected from CD11b⁺/CD14⁻/CD15⁺, CD11b⁺/CD14⁺/CD15⁺, Lin⁻/DR⁻, CD34⁺/CD33⁺/CD13⁺, ARG⁺/CD14⁺, CD34⁺/Lin⁻ /DR⁻/CD11b⁺/CD15⁺, CD14⁺/HLA-DR⁻/low, or Lin⁻/HLA-DR⁻/low/CD11b⁺/CD33⁺; and the pro-inflammatory CD14⁺/CD16⁺ cells.

The kit of the invention further comprises antibodies against each one of said cell types, as well as reagents required for the detection of those antibodies. The antibodies may be either monoclonal or polyclonal, but they are preferably monoclonal antibodies. Both the antibodies and the reagents provided are used for measuring the levels of the cell types listed, in said blood sample.

As defined by both of the non-therapeutic methods of the invention, the level measured for each one of the cell types listed is compared with a range level of said cell type in blood samples of age-matched controls so as to evaluate whether the level measured is higher or lower than, or within, the normal range level of said cell type, i.e., the range level of said cell type in blood samples of age-matched controls.

As explained above, in case an individual is tested for ALS, these data are used for the preparation of a test profile, which is then compared with a reference profile, optionally included in the kit, expressing a representative relative level of each one of the cell types in blood samples of ALS patients, so as to determine whether said individual has a higher likelihood of having ALS than said age-matched controls. Alternatively, i.e., in case a blood sample taken from an ALS patient is tested, these data may be compared with data obtained from the same patient at a previous or later instant, so as to determine whether the treatment for ALS given to said patient is efficient.

The invention will now be illustrated by the following non-limiting Examples.

### Examples

### Materials and Methods

Patients:

The patient's group included individuals, both males and females, which have been clinically diagnosed as suffering from amyotrophic lateral sclerosis (ALS) and agreed to sign on the informed consent. The control group included male and female volunteers without clinical symptoms of ALS, who agreed to sign on the informed consent.

Whole Blood FACS Staining:

50 μl of whole blood samples were incubated with 5 μl of each of the designated mAb (see below) for 45 minutes at 4° C. Two ml of FACSlyse (Becton Dickinson, San Jose, Calif.) was added to each tube, and the tubes were then incubated at room temperature for 12 min, followed by wash with 2 ml PBS. From each sample, 10⁵ events were acquired by FACSCalibur (Becton Dickinson, San Jose, Calif.) and analyzed by the FCS Express V3 software.

The Designated Monoclonal Antibodies (mAb's):

CD3, CD4, CD8, CD14, CD15, CD11b, CD16, Lin, HLA-DR, CD33, TCRgd—Becton Dickinson, San Jose, Calif. TLR4 eBioscience San Diego Calif.

### Example 1

### ALS Patients Show Elevated Level of CD11b⁺/CD14⁻ Cells in PBMCs Compared with Alzheimer's Patients and Healthy Controls

Myeloid suppressor cells constitute a population of immature myeloid cells with potent immunosuppressive functions. These cells have been shown to infiltrate tumors and to regulate adaptive immune responses to cancer cells in experimental animals and human cancer patients. They can induce immunosuppression under normal, inflammatory or surgical/traumatic stress conditions. The accumulation of myeloid suppressor cells is one of the major mechanisms of tumor escape (Frey, 2006; Serafini et al., 2006a; Bunt et al., 2006; Makarenkova et al., 2006). Myeloid suppressor cells are of interest because they have the ability to suppress T-cell immune responses by a variety of mechanisms (Sica and Bronte, 2007; Serafini et al., 2006a; Talmadge, 2007; Nagaraj and Gabrilovich, 2007). These cells are heterogeneous cellular population containing macrophages, granulocytes, immature dendritic cells and early myeloid precursors.

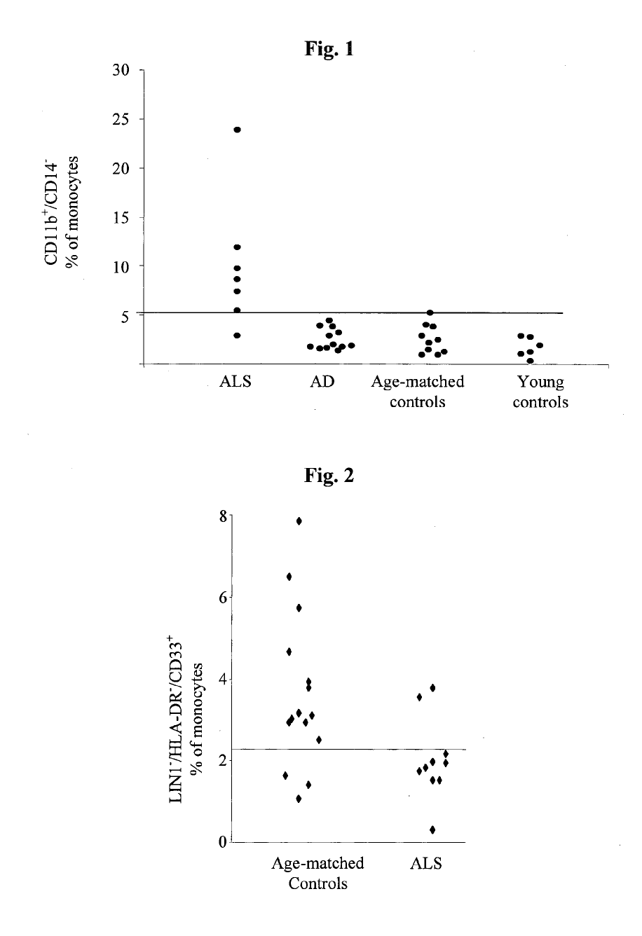

In this study, the level of CD11b⁺/CD14⁻ myeloid derived suppressor cells (MDSCs) in the blood of ALS patients was compared with that of Alzheimer's patients, age-matched controls and young adult (age 20-50 years) controls. In particular, whole blood sample of ALS patients, Alzheimer's patients, age-matched controls and young controls (n=7, 12, 10 and 6, respectively) were stained with monoclonal antibodies against CD14 and CD11b; and the percentage of CD11b⁺/CD14⁻ cells out of total monocytes was determined by FACS. As shown in FIG. 1, the percentage of CD11b⁺/CD14⁻ cells out of total monocytes in ALS patients was significantly higher compared to age-matched controls, young controls and Alzheimer's disease patients. The elevated level of myeloid suppressor cells found in the peripheral blood of ALS patients restricts the reparative T-cell immune response and thus allows the toxic inflammation induced by the microglia to spread in the tissue.

### Example 2

### ALS Patients Show Elevated Level of Lin⁻/DR⁻/CD33⁺ Cells in PBMCs Compared with Healthy Controls

Since the myeloid cell population contains many different cell types and myeloid cell differentiation is a continuum of processes, MDSCs may display diverse phenotypic markers that reflect the spectrum of immature to mature myeloid cells.

In this study we show that the level of Lin⁻/DR⁻/CD33⁺ cells, i.e., a phenotype of MDSC different than that shown in Example 1, in the blood of ALS patients is elevated as well. In particular, whole blood sample of ALS patients and healthy controls (n=15 and 10, respectively) were stained with monoclonal antibodies against Lin, HLA-DR and CD33; and the percentage of Lin⁻/HLA-DR⁻/CD33⁺ cells out of total monocyte population for each patient was determined by FACS. As shown in FIG. 2, the percentage of Lin⁻/HLA-DR⁻/CD33⁺ myeloid cells out of total monocytes in

ALS patients was significantly higher compared to healthy controls.

It was found that the frequencies of CD33⁺HLA-DR⁻ MDSC isolated from the peripheral blood of patients with metastatic renal cell carcinoma are significantly elevated compared with CD33⁺HLA-DR⁻ cells from healthy donors. As further found, MDSC isolated from the peripheral blood of renal cell carcinoma patients, but not from healthy donors, were capable of suppressing antigen-specific T-cell responses in vitro through the secretion of reactive oxygen species and nitric oxide upon interaction with cytotoxic T lymphocytes (CTLs) (Kusmartsev et al., 2008).

### Example 3

### ALS Patients Show Elevated Level of Gamma-Delta T-Cells

Gamma-delta (γδ) T cells represent a small subset of T cells possessing a distinct T cell receptor (TCR) on their surface. These cells are implicated in host defense against microbes and tumors but their mode of function remains largely unresolved.

A variety of sometimes-conflicting effector functions have been ascribed to these cells depending on their tissue distribution, antigen-receptor structure and local microenvironment. In particular, they were shown to play a role in immunosurveillance and immunoregulation (Girardi, 2006), and were found to be an important source of IL-17 (Roark et al., 2008) and to induce robust CD8⁺ cytotoxic T cell response (Brandes et al., 2009).

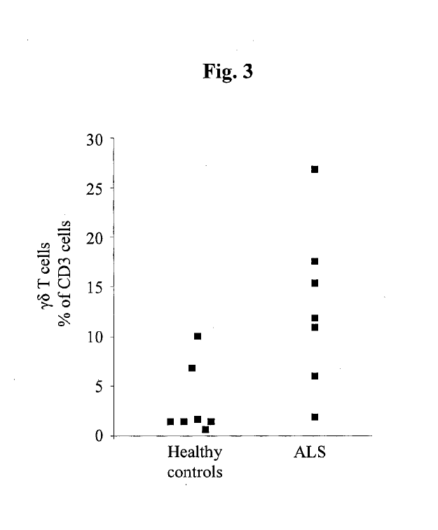

In this study, the level of γδ T cells in PBMCs of ALS patients was compared with that in PBMCs of healthy controls. In particular, freshly isolated PBMCs of ALS patients and healthy controls (n=7 in each group) were double-stained with monoclonal antibodies against CD3 and with monoclonal antibodies against γδ T cell receptor, and the percentage of γδ T cells out of total CD3 cells was determined by FACS. As shown in FIG. 3, the percentage of γδ T cells out of total CD3⁺ cells in ALS patients was significantly higher than that in healthy controls, indicating that this unique cell subset can also be used as a biological marker for ALS.

### REFERENCES

[No authors listed] Anti-inflammatory drugs fall short in Alzheimer's disease, Nat Med., 2008, 14(9), 916
Beers D. R., Henkel J. S., Xiao Q., Zhao W., Wang J., Yen A. A., Siklos L., McKercher S. R., Appel S. H., Wild-type microglia extend survival in PU.1 knockout mice with familial amyotrophic lateral sclerosis, Proc Natl Acad Sci USA, 2006, 103, 16021-16026
Beers D. R., Henkel J. S., Zhao W., Wang J., Appel S. H., CD4⁺ T cells support glial neuroprotection, slow disease progression, and modify glial morphology in an animal model of inherited ALS, Proc Natl Acad Sci USA, 2008, 105(40), 15558-15563
Boillee S., Yamanaka K., Lobsiger C. S., Copeland N. G., Jenkins N. A., Kassiotis G., Kollias G., Cleveland D. W., Onset and progression in inherited ALS determined by motor neurons and microglia, Science, 2006, 312, 1389-1392
Brandes M., Willimann K., Bioley G., Lévy N., Eberl M., Luo M., Tampé R., Lévy F., Romero P., Moser B., Cross-presenting human gammadelta T cells induce robust CD8⁺ alphabeta T cell responses, Proc Natl Acad Sci USA, 2009, 106(7), 2307-2312
Bunt S. K., Sinha P., Clements V. K., Leips J., Ostrand-Rosenberg S., Inflammation induces myeloid-derived suppressor cells that facilitate tumor progression, J Immunol., 2006, 176(1), 284-290
Butovsky O., Hamaoui M. K., Kunis G., Ophir E., Landa G., Cohen H., Schwartz M., Glatiramer acetate fights against Alzheimer's disease by inducing dendritic-like microglia expressing insulin-like growth factor 1, Proc Natl Acad Sci USA, 2006, 103(31), 11784-11789
Cardona A. E., Pioro E. P., Sasse M. E., Kostenko V., Cardona S. M., Dijkstra I. M., Huang D., Kidd G., Dombrowski S., Dutta R., Lee J. C., Cook D. N., Jung S., Lira S. A., Littman D. R., Ransohoff R. M., Nat Neurosci., 2006, 9(7), 917-924
Chiu I. M., Chen A., Zheng Y., Kosaras B., Tsiftsoglou S. A., Vartanian T. K., Brown R. H. Jr, Carroll M. C., T lymphocytes potentiate endogenous neuroprotective inflammation in a mouse model of ALS, Proc Natl Acad Sci USA, 2008, 105(46), 17913-17918
Clement A. M., Nguyen M. D., Roberts E. A., Garcia M. L., Bake S., Rule M., McMahon A. P., Doucette W., Siwek D., Ferrante R. J., Brown R. H., Julien Jr. J-P., Goldstein L. S. B., Cleveland D. W., Wild-type nonneuronal cells extend survival of SOD1 mutant motor neurons in ALS mice, Science, 2003, 302, 113-117
Corti S., Locatelli F., Donadoni C., Guglieri M., Papadimitriou D., Strazzer S., Del Bo R., Comi G. P., Wild-type bone marrow cells ameliorate the phenotype of SOD1-G93A ALS mice and contribute to CNS, heart and skeletal muscle tissues, Brain, 2004, 127(11), 2518-2532
Dunn G. P., Bruce A. T., Ikeda H., Old L. J., Schreiber R. D., Cancer immunoediting: from immunosurveillance to tumor escape, Nat Immunol, 2002, 3(11), 991-998
Etminan M., Carleton B. C., Samii A., Non-steroidal anti-inflammatory drug use and the risk of Parkinson disease: a retrospective cohort study, J Clin Neurosci., 2008, 15(5), 576-577
Frey A. B., Myeloid suppressor cells regulate the adaptive immune response to cancer, J Clin Invest., 2006, 116(10), 2587-2590
Frey A. B., Monu N., Signaling defects in anti-tumor T cells, Immunol Rev., 2008, 222, 192-205
Gendelman H. E., J Neurovirol., 2002, 8(6), 474-479
Girardi M., Immunosurveillance and immunoregulation by gammadelta T cells, J Invest Dermatol., 2006, 126(1), 25-31
Gordon P. H., Moore D. H., Miller R. G., Florence J. M., Verheijde J. L., Doorish
C., Hilton J. F., Spitalny G. M., MacArthur R. B., Mitsumoto H., Neville H. E., Boylan K., Mozaffar T., Belsh J. M., Ravits J., Bedlack R. S., Graves M. C., McCluskey L. F., Barohn R. J., Tandan R., Western ALS Study Group. Efficacy of minocycline in patients with amyotrophic lateral sclerosis: a phase III randomised trial, Lancet Neurol., 2007, 6(12), 1045-1053
Gowing G., Philips T., Van Wijmeersch B., Audet J. N., Dewil M., Van Den Bosch L., Billiau A. D., Robberecht W., Julien J. P., Ablation of proliferating microglia does not affect motor neuron degeneration in amyotrophic lateral sclerosis caused by mutant superoxide dismutase, J Neurosci, 2008, 28, 10234-10244
Graves M. C., Fiala M., Dinglasan L. A., Liu N. Q., Sayre J., Chiappelli F., van Kooten C., Vinters H. V., Inflammation in amyotrophic lateral sclerosis spinal cord and brain is mediated by activated macrophages, mast cells and T cells, Amyotroph Lateral Scler Other Motor Neuron Disord., 2004, 5(4), 213-219
Hauben E., Nevo U., Yoles E., Moalem G., Agranov E., Mor F., Akselrod S., Neeman M., Cohen I. R., Schwartz M., Lancet, 2000, 355(9200), 286-287
Hauben E., Gothilf A., Cohen A., Butovsky 0., Nevo U., Smirnov I., Yoles E., Akselrod S., Schwartz M., J Neurosci., 2003, 23(25), 8808-8819
Huang H., et al., [Effect of transplantation of wild-type bone marrow stem cells in mouse model of familial amyotrophic lateral sclerosis, Zhongguo Yi Xue Ke Xue Yuan Xue Bao, 2006, 28 (4), 562-566]
Henkel J. S., Beers D. R., Siklos L., Appel S. H., The chemokine MCP-1 and the dendritic and myeloid cells it attracts are increased in the mSOD1 mouse model of ALS, Mol Cell Neurosci, 2006, 31(3), 427-437
Karman J., Ling C., Sandor M., Fabry Z., Initiation of immune responses in brain is promoted by local dendritic cells, J Immunol, 2004, 173(4), 2353-2361
Kipnis J., Yoles E., Schori H., Hauben E., Shaked I., Schwartz M., J Neurosci., 2001, 21(13), 4564-4571
Kipnis J., Mizrahi T., Hauben E., Shaked I., Shevach E., Schwartz M., Proc Natl Acad Sci USA, 2002, 99(24), 15620-15625
Kusmartsev S., Su Z., Heiser A., Dannull J., Eruslanov E., Kiibler H., Yancey D., Dahm P., Vieweg J., Reversal of myeloid cell-mediated immunosuppression in patients with metastatic renal cell carcinoma, Clin Cancer Res., 2008, 14(24), 8270-8278
Laurie C., Reynolds A., Coskun O., Bowman E., Gendelman H. E., Moslry R. L., CD4⁺ T cells from Copolymer-1 immunized mice protect dopaminergic neurons in the 1-methyl-4-phenyl-1,2,3,6-tetrahydropyridine model of Parkinson's disease, J Neuroimmunol, 2007, 183(1), 60-68
Ling C., Sandor M., Suresh M., Fabry Z., Traumatic injury and the presence of antigen differentially contribute to T-cell recruitment in the CNS, J Neurosci, 2006, 26(3), 731-741
Makarenkova V. P., Bansal V., Matta B. M., Perez L. A., Ochoa J. B., CD11b+/Gr-1+ myeloid suppressor cells cause T cell dysfunction after traumatic stress, J Immunol., 2006, 176(4), 2085-2094
Marchetti B., Serra P. A., Tirolo C., L'episcopo F., Caniglia S., Gennuso F., Testa N., Miele E., Desole S., Barden N., Morale M. C., Brain Res Rev., 2005, 48(2), 302-321
Moalem G., Leibowitz-Amit R., Yoles E., Mor F., Cohen I. R., Schwartz M., Nat Med., 1999, 5(1), 49-55
Moisse K., Strong M. J., Innate immunity in amyotrophic lateral sclerosis, Biochim Biophys Acta., 2006, 1762(11-12), 1083-1093
Nagaraj S., Gabrilovich D. I., Myeloid-derived suppressor cells, Adv Exp Med Biol., 2007, 601, 213-223
Nagaraj S., Gabrilovich D. I., Tumor escape mechanism governed by myeloid-derived suppressor cells, Cancer Res., 2008, 68(8), 2561-2563
Nevo U., Kipnis J., Golding I., Shaked I., Neumann A., Akselrod S., Schwartz M., Trends Mol Med., 2003, 9(3), 88-93
Ochoa A. C., Zea A. H., Hernandez C., Rodriguez P. C., Arginase, prostaglandins, and myeloid-derived suppressor cells in renal cell carcinoma, Clin Cancer Res., 2007, 13(2 Pt 2), 721s-726s
Rapalino O., Lazarov-Spiegler O., Agranov E., Velan G. J., Yoles E., Fraidakis M., Solomon A., Gepstein R., Katz A., Belkin M., Hadani M., Schwartz M., Nat Med., 1998, 4(7), 814-821
Roark C. L., Simonian P. L., Fontenot A. P., Born W. K., O'Brien R. L., Gammadelta T cells: an important source of IL-17, Curr Opin Immunol., 2008, 20(3), 353-357
Rolls A., Avidan H., Cahalon L., Schori H., Bakalash S., Litvak V., Lev S.,
Lider O., Schwartz M., A disaccharide derived from chondroitin sulphate proteoglycan promotes central nervous system repair in rats and mice, Eur J Neurosci, 2004, 20(8), 1973-1983
Rosen D. R., Mutations in Cu/Zn superoxide dismutase gene are associated with familial amyotrophic lateral sclerosis, Nature, 1993, 364, 362
Sargsyan S. A., Monk P. N., Shaw P. J., Microglia as potential contributors to motor neuron injury in amyotrophic lateral sclerosis, Glia., 2005, 51(4), 241-253
Schori H., Yoles E., Schwartz M., T-cell-based immunity counteracts the potential toxicity of glutamate in the central nervous system, J. Neuroimmunol., 2001, 119(2), 199-204
Schwartz M., Autoimmunity as the body's defense mechanism against the enemy within: Development of therapeutic vaccines for neurodegenerative disorders, J Neurovirol., 2002, 8(6), 480-485
Schwartz M., Hauben E., Science, 2002, 296(5572), 1400 Schwartz M., Kipnis J., Trends Immunol., 2002, 23(11), 530-534 [Erratum in: Trends Immunol., 2003, 24(1), 12]
Schwartz M., Shaked I., Fisher J., Mizrahi T., Schori H., Trends Neurosci., 2003, 26(6), 297-302
Seksenyan A., Ron-Harel N., Azoulay D., Cahalon L., Cardon M., Rogeri P., Ko M. K., Weil M., Bulvik S., Rechavi G., Amariglio N., Konen E., Koronyo-Hamaoui M., Somech R., Schwartz M., Thymic involution in amyotrophic lateral sclerosis, J Cell Mol Med., 2009,
Serafini P., Borrello I., Bronte V., Myeloid suppressor cells in cancer: recruitment, phenotype, properties, and mechanisms of immune suppression, Semin Cancer Biol., 2006a, 16(1), 53-65
Serafini P., Meckel K., Kelso M., Noonan K., Califano J., Koch W., Dolcetti L., Bronte V., Borrello I., Phosphodiesterase-5 inhibition augments endogenous antitumor immunity by reducing myeloid-derived suppressor cell function, J Exp Med., 2006b, 203(12), 2691-2702
Shaked I., Porat Z., Gersner R., Kipnis J., Schwartz M., J Neuroimmunol., 2004, 146(1-2), 84-93
Shaked I., Tchoresh D., Gersner R., Meiri G., Mordechai S., Xiao X., Hart R. P., Schwartz M., J Neurochem., 2005, 92(5), 997-1009
Shechter R., London A., Varol C., Raposo C., Cusimano M., Yovel G., Rolls A., Mack M., Pluchino S., Martino G., Jung S., Schwartz M., Infiltrating blood-derived macrophages are vital cells playing an anti-inflammatory role in recovery from spinal cord injury in mice, PLoS Med., 2009, 6(7)
Sica A., Bronte V., Altered macrophage differentiation and immune dysfunction in tumor development, J Clin Invest., 2007, 117(5), 1155-1166
Simard A. R., Soulet D., Gowing G., Julien J. P., Rivest S., Bone marrow-derived microglia play a critical role in restricting senile plaque formation in Alzheimer's disease, Neuron, 2006, 49(4), 489-502
Smyth M. J., Dunn G. P., Schreiber R. D., Cancer immunosurveillance and immunoediting: the roles of immunity in suppressing tumor development and shaping tumor immunogenicity, Adv Immunol, 2006, 90, 1-50
Suzuki E., Kapoor V., Jassar A. S., Kaiser L. R., Albelda S. M., Gemcitabine selectively eliminates splenic Gr-1+/CD11b+ myeloid suppressor cells in tumor-bearing animals and enhances antitumor immune activity, Clin Cancer Res., 2005, 11(18), 6713-6721
Taams L. S., Akbar A. N., Curr Top Microbiol Immunol., 2005, 293, 115-131
Talmadge J. E., Pathways mediating the expansion and immunosuppressive activity of myeloid-derived suppressor cells and their relevance to cancer therapy, Clin Cancer Res., 2007, 13(18 Pt 1), 5243-5248
Weydt P., Yuen E. C., Ransom B. R., Moller T., Increased cytotoxic potential of microglia from ALS-transgenic mice, Glia, 2004, 48(2), 179-182
Yoles E., Hauben E., Palgi O., Agranov E., Gothilf A., Cohen A., Kuchroo V., Cohen I. R., Weiner H., Schwartz M., J Neurosci., 2001, 21(11), 3740
Zea A. H., Rodriguez P. C., Atkins M. B., Hernandez C., Signoretti S., Zabaleta J., McDermott D., Quiceno D., Youmans A., O'Neill A., Mier J., Ochoa A. C., Arginase-producing myeloid suppressor cells in renal cell carcinoma patients: a mechanism of tumor evasion, Cancer Res., 2005, 65(8), 3044-3048
Ziv Y., Avidan H., Plichino S., Martino G., Schwartz M., Synergy between immune cells and adult neural stem/progenitor cells promotes functional recovery from spinal cord injury, Proc Natl Acad Sci USA, 2006, 103(35), 13174-13179
Ziv Y., Finkelstein A., Geffen Y., Kipnis J., Smirnov I., Shpilman S., Vertkin I., Kimron M., Lange A., Hecht T., Reyman K. G., Marder J. B., Schwartz M., Yoles E., A novel immune-based therapy for stroke induces neuroprotection and supports neurogenesis, Stroke, 2007, 38 (2 Suppl), 774-782

1. A method for determining the efficacy of a treatment for ALS in an ALS patient, comprising:
(i) measuring the level of at least one cell type selected from regulatory T-cells, gamma-delta (γδ) T-cells, myeloid derived suppressor cells (MDSCs) or natural killer cells in a peripheral blood sample obtained from said patient at two consecutive instants, the earlier of said instants is prior to or during said treatment and the later of said instants is during said treatment; and
(ii) comparing the levels measured for each one of said at least one cell type at said two instants,
wherein an alteration of the level measured for one or more of said at least one cell type at said later instant compared with the level measured for said cell type at said earlier instant towards a reference level representing a range level of said cell type in blood samples of age-matched controls is correlated with the efficacy of said treatment.

2. The method of claim 1, wherein the earlier of said instants is prior to or during said treatment and the later of said instants is about 1, 2, 3, 4, 5, 6 months or more later than the earlier instant.

3. A method for treatment of an ALS patient comprising administering to said patient an effective amount of an agent capable of reducing myeloid derived suppressor cell level in peripheral blood.

4. The method of claim 3, wherein said agent capable of reducing myeloid derived suppressor cell level in a peripheral blood is gemcitabine, sildenafil, tadalafil or vardenafil.

5. The method of claim 3, further comprising administering to said patient an effective amount of an agent capable of augmenting level of anti-self T-cells in a peripheral blood, autologous T cells and/or activated T cells.

6. The method of claim 5, wherein said agent capable of augmenting level of anti-self T-cells in a peripheral blood is glatiramer acetate (Copaxone®).

7. A kit for diagnosing the likelihood of ALS in a tested individual; or for determining the efficacy of a treatment for ALS in an ALS patient, said kit comprising:
(i) a list of cell types selected from regulatory T-cells, gamma-delta (γδ) T-cells, pro-inflammatory monocytes, myeloid derived suppressor cells (MDSCs), or natural killer cells;
(ii) antibodies against each one of said cell types;
(iii) reagents for detecting said antibodies;
(iv) a list of reference levels representing range levels of said cell types in blood samples of age-matched controls;
(v) optionally a reference profile expressing a representative relative level of each one of said cell types in blood samples of ALS patients; and
(vi) instructions for use.
